(19) United States
(12) Patent Application Publication
(10) Pub. No.: US 2005/0010229 A1
(43) Pub. Date: Jan. 13, 2005

(54) Arrangement and method for supplying implant fixtures made principally of titanium

(76) Inventor: Jan Hall, Goteborg (SE)

Correspondence Address: CONNOLLY BOVE LODGE & HUTZ LLP, SUITE 800, 1990 M STREET NW, WASHINGTON, DC, 20036-3425, US

(21) Appl. No.: 10/482,731

(22) PCT Filed: Jun. 26, 2002
(86) PCT No.: PCT/SE02/01256

(30) Foreign Application Priority Data
Jul. 4, 2001 (SE) 0102390-2

(51) Int. Cl. A61B 17/56

(52) U.S. Cl. 606/76

(57) ABSTRACT

In an arrangement and method for supplying implant fixtures (2) made of titanium with surface structures and added bone-growth-stimulating agents or substances, the fixtures are prepared for selective use in different implantation situations. One or more first devices (9) are intended, by means of surface treatments, for example in the form of etching, shot-peening, plasma spraying and/or electrochemical treatment (so-called anodic oxidation), to supply fixtures (13-20) with different surface structures. One or more second devices (21) are intended to apply calcium phosphate layers in different dose qpplications onto implant fixtures divided into different ranges (partial ranges) (11, 12), which dose applications create layer thicknesses of the order of 1 nanometer to 3000 nanometers. In addition, as an alternative or as a complement to this, one or more third devices (22) can be provided to treat implant fixtures belonging to said range, or another range (partial range) which lacks CaP layers, by applying bone-growth-stimulating substance, for example rhBMP-2 or rhBMP-7, in different dose applications with different quantity and strength. A very high degree of selectivity is achieved in this way.

The present invention relates to an arrangement for supplying implant fixtures which are made principally of titanium and which, as a function of allocated surface structures and added bone-growth-stimulating or bone-growth-maintaining agents or substances, are prepared for selective use in different implantation situations or implantation cases. The invention also relates to a method for supplying implant fixtures made preferably of titanium and with different surface structures and added bone-growth-stimulating agents or substances.

It is already quite generally known to provide implant fixtures with porous oxide layers and to coat fixtures, machined or provided with said porous layers, with bone-growth-stimulating agents or substances. In this connection, reference can be made to relevant parts of the applications WO 98/48862, 9901971-3, 9901973-9, 9901974-7, 0001201-3 and 0001202-1 submitted by the same Applicant as submits the present patent application. Reference is also made to the prior art cited in these applications and also to the references mentioned in connection with these.

In the prior art, there is often a need to be able to individualize the anchoring of implants so that optimum treatment results are achieved with the fewest possible, but effective, measures. The invention aims to go further into this problem and proposes improvements which afford very good results.

The aim is to be able to use known and proven specialist skills and known and proven methods and devices for implant fixtures as such. There is a large range of different fixture structures and components and these do not need to be increased in number. The invention is also aimed at solving this problem.

There is a need to establish effective cooperation between the parties involved, such as patient, dentist, surgeon, radiologist, manufacturer, etc. Different skills must be able to be brought together and used effectively, and the ordering and production methods are to be IT-controlled to the greatest possible extent. Thus, for example, it has been found that the quantity of bone-growth-stimulating substance or agent in the different individual dental cases is dependent not only on the individual, but also on the position of the treatment site in the jaw bone, i.e. the rear parts of the jaw bone may need greater quantities than the outer parts, or vice versa. There is therefore a need to permit more sophisticated choice of the quantity of substance. The invention solves this problem too.

It is important that the arrangement and the method in connection with production of fixtures and supply of fixtures must be able to be based on and targeted toward results which are obtained in research and development in the field of bone growth stimulation and the body's ability to respond to such agents and substances. The invention is based on these aspects and proposes a dose application arrangement which can be optimized with regard to growth, incorporation times, tooth position, etc.

That which can mainly be regarded as characterizing an arrangement according to the invention is, inter alia, that one or more first devices are intended, by means of surface treatments in the form of etching, shot-peening, plasma spraying and/or electrochemical treatment/anodic oxidation, to supply fixtures with different surface structures and/or oxide layers provided with porosities. These features are combined with one or both of the following alternatives. In the first alternative, one or more second devices are provided which are intended to apply calcium phosphate layers in different dose applications onto the implant fixtures, which dose applications create CaP layers within the range of 1 nanometer to 3000 nanometers. In the second alternative, one or more third devices are used which are intended to treat implant fixtures, with or without said CaP layers, by applying bone-growth-stimulating substance, for example rhBMP-2, rhBMP-7, rhBMP-14, etc., in different dose applications, for example in dose applications obtained by means of about 0.05-50 μg, preferably 5-20 μG, of bone-growth-stimulating substance, or rhBMP. In the term rhBMP, the letters BMP stand for bone morphogenetic proteins. The different numbers after this term relate to different substances within the TGF-β superfamily.

In embodiments of the arrangement, the first devices treat the surfaces either by means of mechanical devices so as to perform mechanical working of the surfaces. The first devices can alternatively surface-treat the surfaces so that porous oxide layers are established, it being possible to obtain more or less open pores in the oxide layer. In one embodiment, the third devices can be intended to charge the fixture surfaces with, for example, 5 μg of bone-growth-stimulating substance, for example some form of rhBMP such as rhBMP-2. The arrangement causes retention of ca. 2-2.5% to ca. 4.5-5% bone-growth-stimulating substance (rhBMP-2) on the fixture surface after the surfaces have been washed with buffer. The concentration of bone-growth-stimulating substance (rhBMP-2) can be between ca. 0.5% and ca. 2.5% after ca. two weeks of incorporation in said buffer, so-called post-loading. It can be noted that the surface structures provided with porous layers store quantities of bone-growth-stimulating substance (rhBMP-2) which lie in the upper range, and those with only machined surface structures store quantities of bone-growth-stimulating substance (rhBMP-2) which lie in the lower range. In other embodiments, the surface structure can be charged with a dose of ca. 10 μg of bone-growth-stimulating substance (rhBMP-2) or a dose of ca. 20 μg. In a further embodiment, implant fixtures can have a porous oxide layer with more or less open pores charged with CaP layers of ca. 100 nanometers and thereafter treated with ca. 5 μg of bone-growth-stimulating substance.

In a method according to the above, fixtures with different surface structures and/or oxide layers provided with porosities are supplied by means of surface treatment in the form of etching, shot-peening, plasma spraying, treatment by wet chemistry or laser, and/or electrochemical treatment. According to the novel method, some of the implant fixtures are provided with calcium phosphate layers (CaP layers) by different dose applications which create CaP layers of the order of 10-3000 nanometers. As an alternative or as a complement to this, one or more of said implant fixtures, or one or more of second implant fixtures which lack CaP layers/bone-growth-stimulating substance (for example rhBMP-2), can be applied in different dose applications by means of one or more third devices. Said dose applications are obtained by means of ca. 5-20 μg (rhBMP).

An arrangement for supplying implant fixtures made of titanium with surfaces provided with different surface structures and coated with bone-growth-stimulating and/or bone-growth-maintaining agent or substance is mainly characterized by, inter alia, a device which defines surface structures and a dose or doses of agent or substance on or in the respective implant fixture, and ordering equipment for making available implant fixtures with defined surface structures and doses. The arrangement is further characterized by equipment for receiving ordered implant fixture(s) and by production equipment for producing implant fixtures with defined surface structure. Finally, the arrangement is characterized by application equipment for applying layers or doses of CaP and/or bone-growth-stimulating substance (rhBMP-2) on the surface structure or surface structures of the respective implant.

By means of what has been proposed above, desired treatment principles can be used, for example in the dental field. The dentist or equivalent can analyze the jaw bone situation and the tooth situation and propose a specific fixture configuration and substance quantity in accordance with the above. The ordering function can be effectively established, and the dentist or equivalent can if appropriate be offered access to different fixtures. Well proven production techniques for production of porosities and fixtures with different types of surfaces can be offered. Dose and layer application can be carried out in a known manner by means of immersion methods, sputtering methods, heat treatment for different degrees of crystallization of the CaP layers, etc. After production of the fixture or fixtures has been completed, they can be sent back to the orderer (dentist, surgeon, etc.).

A presently proposed arrangement and method will be described below with reference to the attached drawings, in which:

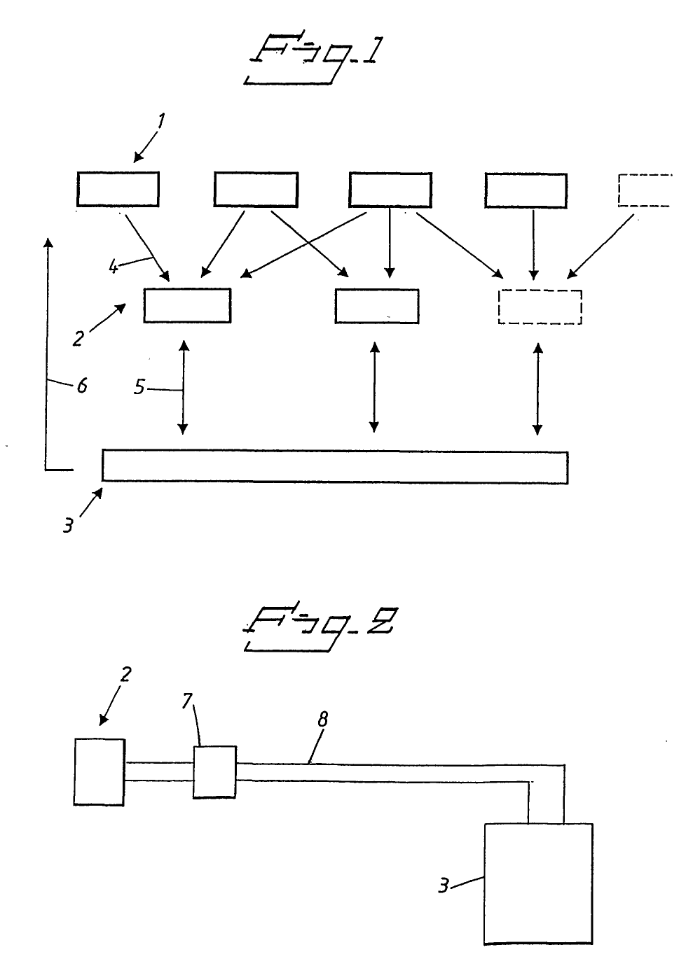

FIG. 1 shows a flow diagram between the parties involved in supplying implant fixtures,

FIG. 2 shows, in block diagram form, an arrangement for ordering the implant fixture in question, where the orderer places the order with a manufacturer,

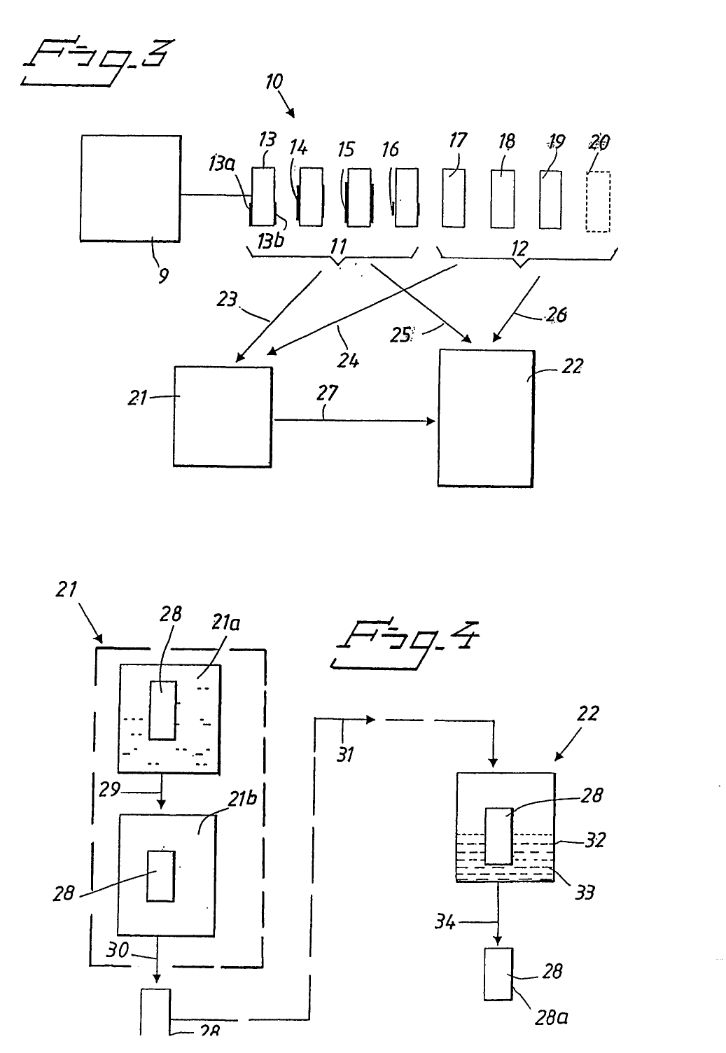

FIG. 3 shows, in block diagram form, the production of the ordered fixtures, which can be divided into two or more groups with regard to surface structure and assigned to treatments for CaP application and for coating with bone-growth-stimulating substance,

FIG. 4 shows a flow diagram for an implant or a fixture which is to be coated with CaP and/or bone-growth-stimulating substance.

FIG. 1 shows an arrangement involving three parties. The first party can be the patients or clients 1 who are to undergo the treatment in question with an implant fixture. The clients 1 can turn to a second party 2 which can be the dentist, surgeon, radiology unit, etc. The third party 3 can be the manufacturer who will produce the implant fixtures with which the patients 1 are to be fitted. The patients or clients can turn to dentists, surgeons, etc., in accordance with arrows 4. The second party can cooperate with the production party, as has been illustrated by two-way arrows 5. If appropriate, a certain feedback or exchange of information can take place between the clients and the producer, as has been illustrated by the broken arrow 6.

When the second party has analyzed and defined the type of fixture which is to be used in the particular treatment case on the particular patient, he or she can place an order with the party 3. This order can be placed via ordering equipment 7 which can consist of computerized ordering equipment. The order can be placed via the public telecommunications or computer network, as is illustrated by 8. Said network can include all or part of the public telecommunications network, the Internet, etc. Order routines of a type known per se can be used, and the ordering functions can likewise take place wholly or partly via the network.

FIG. 3 shows a first device 9 at the manufacturer 3 (cf. FIG. 1). By means of the first device 9, it is possible to produce implants 10 in a manner known per se which can be divided into two or more ranges, and in FIG. 3 two different ranges of implants have been shown by 11 and 12. In the first range 11 of implants or fixtures 13, 14, 15, 16, these can be provided with porous layers 13a which can be different in respect of thickness, more or less open pores, etc. The fixtures in the range 12 can be of the type which lacks marked porosity, i.e. they have only been machined. The last-mentioned implants have been shown by 17, 18, 19 and 20. Different degrees of machining can be used for the surfaces of the implants, which surfaces can thus have different degrees of finishing or topographies.

The implants in question are preferably made of titanium and the layers on the implants or fixtures in the range 11 are thus titanium oxide layers.

Since the treatment of fixture surfaces is well known per se, it will not be described in detail here. It will be noted, however, that in electrochemical oxidation a vessel is used in which the implant or implant parts in question are immersed in an electrolyte. The implant is connected to a voltage source, and the surface layers of the fixture or implant and its thickness and pore composition can be determined by the type of electrolyte, the treatment time, and other parameters which are known per se in this connection.

FIG. 3 also shows a second device 21 in which dose application of CaP can be carried out. In addition, FIG. 3 shows a third device 22 in which application or coating of bone-growth-stimulating substance can be carried out. The implants according to the groups 11 and 12 can alternatively be provided with CaP layers or bone-growth-stimulating substance layers, for example rhBMP-2 layers, in accordance with the arrows 23, 24 and 25, 26, respectively. In one embodiment, the implants or fixtures can be provided with both types of layers, in which case the fixtures in question are first provided with CaP layers and then, on top of this CaP layer or these CaP layers, with the bone-growth-stimulating substance layer in question. This has been illustrated by the arrow 27.

FIG. 4 shows two different functional stages in the second device 21. In the first stage, which has been indicated by 21a, the fixture or implant is treated in a unit in which RF sputtering can be carried out. This method is known per se and will therefore not be described in detail here; it will simply be noted that the fixture 28 is coated with applied CaP layers. These are applied as a function of any masking if only part of the surfaces (e.g. outer surfaces) of the implant is to be coated. The implant or fixture thus coated is transferred to a heating oven 21b in the next stage, in which the implant is crystallized in a manner known per se or is given the desired degree of crystallization. The transfer of the implant 28 from the unit 21a to the unit 21b takes place in the direction of the arrow 29. The finished, heat-treated implant is transferred from the oven 21b in the direction of the arrow 30. If the implant 28 is also to be treated with bone-growth-stimulating substance, it is transferred in the direction of the arrow 31 to a third device 22 which includes a vessel 32 with a solution 33 of bone-growth-stimulating substance. After the treatment, the finished implant or the finished fixture 28 is obtained, as has been illustrated by the arrow 34.

Trials have been carried out investigating the retention function of rhBMP-2 or other selected bone-growth-stimulating agent on a surface with coated CaP layer, on a surface consisting of porous layers with open and partially closed pores, and on a surface that has only been machined, for example milled or turned. Measurements have been conducted on CaP layers of two thicknesses and degrees of crystallization, namely CaP layers of 10-200 nanometers (crystalline) and 1500-3000 nanometers (amorphous layer). It will be noted that the amount of hard tissue, the vascularity and tissue enclosure ought to vary between the respective surface modification and the applied dose of bone-growth-stimulating substance. On application of 5 μg of bone-growth-stimulating substance (rhBMP-2), the surface provided with oxide layers with open pores has the hardest tissue, vascularity and tissue enclosure on the coated surface. CaP 100 nanometers and the oxide layer with partially closed pores ought to have a more moderate amount of hard material, vascularity and tissue enclosure. Hard tissue formation will probably not be present at Cap 2000 nanometers coated on a surface that has only been machined. Surfaces coated with CaP 2000 nanometers have a moderate tissue enclosure and vascularity, while the surface that has only been machined does not. On using 10 μg of bone-growth-stimulating substance (rhBMP-2), hard tissue formation ought to be present on most of the surface in question. The oxide surface with open pores is advantageous and the CaP 2000 nanometer surface also has a hard tissue formation. In this case, there are minimal differences between CaP 100 nanometers, the oxide surface and partially closed pores and the surfaces that have only been machined. A moderate amount of vascularity and tissue encapsulation will probably be observed on the majority of all surfaces. At 20 μg of bone-growth-stimulating substance or rhBMP-2, the CaP 2000 nanometer surfaces appear to generate the hardest tissue formation, vascularity and tissue enclosure.

The effects from dose applications and surface structure configuration can be regarded as significant. Independent analyses in cases with 5, 10 and 20 μg of bone-growth-stimulating substance doses (rhBMP-2) show significant differences and clear trends between the different surface structures. It will be noted that surface-modified and non-surface-modified titanium surfaces which are coated with 5 μg of bone-growth-stimulating substance (rhBMP-2) retain 2% to 5% of bone-growth-stimulating substance (rhBMP-2) after an immediate washing in buffer, and 0.5% to 2.5% of bone-growth-stimulating substance (rhBMP-2) after two weeks in buffer. The surfaces with oxide layers with open pores retain the greatest amount of bone-growth-stimulating substance (rhBMP-2), and the non-modified machined surface retains less bone-growth-stimulating agent. Greater amounts of hard tissue formation, tissue enclosure and vascularity are present on CaP 100 nanometer surfaces and surfaces with oxide layers with closed and open surfaces compared with surfaces modified with Cap 1000 nanometers and machined surfaces. The bone induction is clear on all surfaces which have the least modified or moderate amount of bone tissue formation. There are greater amounts of bone induction, osteoblast cells, osteoid, marrow, tissue enclosure, vascularity and bone against titanium surfaces which have been charged with 20 μg of bone-growth-stimulating substance (BMP-2), followed by a 10 μg dose. This applies also to CaP 100 nanometers and surfaces with oxide layers with open or closed pores at 51g of bone-growth-stimulating substance (rhBMP). There is also significantly greater bone formation and osseointegration on surfaces with CaP 100 nanometers and surfaces with oxide layers with open and closed pores compared with surfaces with CaP 2000 nanometers and machined surfaces with 5 μg of bone-growth-stimulating substance doses (rhBMP doses, for example rhBMP-2 doses). At 10 and 20 μg of bone-growth-stimulating substance (rhBMP, for example rhBMP-2), all surfaces have significant bone formation and osseointegration. Bone-growth-stimulating substance (rhBMP-2) incorporates on surface-modified titanium and generates a bone induction effect. The newly formed bone integrates with the modified surfaces. The bone induction effect is thus dependent on surface and dose and the surfaces with CaP 100 nanometers and oxide layers with open or closed pores constitute the most effective surface modifications. The above details are based on the function and properties of certain selected bone-growth-stimulating substances and are relevant to a large number of bone-growth-stimulating substances.

In FIG. 3, a coating on the fixture 13 is indicated by 13a. The coating can be an oxide layer or a precision-treated surface, which in FIG. 3 is indicated by 13b. An agent or a substance is shown by 28a on the implant/fixture 28 in FIG. 4. This indication too is symbolic. The implants/fixtures can be ordered using the ordering function including surface structure(s), dose amount(s), dose strength(s), etc., and can be ordered on the public telecommunications and computer networks, for example including the Internet.

The invention is not limited to the embodiment shown above by way of example, and instead it can be modified within the scope of the attached patent claims and the inventive concept.

1. An arrangement for supplying implant fixtures (2) which are made principally of titanium and which, as a function of allocated surface structures and added bone-growth-stimulating or bone-growth-maintaining agents or substances, are prepared for selective use in different implantation situations (cases), characterized in that one or more first devices (9) are intended, by means of surface treatments, for example in the form of etching, shot-peening, plasma spraying and/or electrochemical treatment (so-called anodic oxidation), to supply fixtures (13-20) with different surface structures and/or oxide layers provided with porosities, and involving one or both of the following alternatives:
a) one or more second devices (21) are intended to apply calcium phosphate layers (CaP layers) in different dose applications onto implant fixtures divided into or forming different ranges (partial ranges) (11, 12), which dose applications create calcium phosphate layers of the order of 1-3000 nanometers, preferably 100-2000 nanometers, and
b) one or more third devices (22) are provided to treat implant fixtures belonging to one or more of said ranges (partial ranges), or to another range (partial range) which lacks calcium phosphate layers, by applying bone-growth-stimulating substance, for example rhEMP, in different dose applications, for example in dose applications involving about 0.05-50 p.g, preferably 5-20 μg, of bone-growth-stimulating substance, for example rhBMP-2 or rhBMP-7.

2. The arrangement as claimed in patent claim 1, characterized in that the first devices (9) are intended to surface-treat the surfaces by means of mechanical devices which perform mechanical working of the surfaces.

3. The arrangement as claimed in patent claim 1, characterized in that the third devices are intended to coat the relevant fixture surface(s) with ca. 5 μg calcium phosphate layers rhBMP-2 or rhBMP-7.

4. The arrangement as claimed in claim 1, characterized in that it comprises surface structures charged with a dose of less than 50 μg.

5. The arrangement according to claim 1, characterized in that it comprises surface structures with porous oxide layers with more or less open pores charged with CaP layers within the range of 1-3000 nanometers and treated with a dose or doses of bone-growth-stimulating substance.

6. Method for allocating implant fixtures (28) which are made of tissue-compatible material, preferably titanium, and with one or more surface structures and added bone-growth stimulating agents or substances, characterized in that fixtures with different surface structures and/or oxide layers provided with porosities are supplied by means of one or more first devices (9) and
a) implant fixtures are allocated to a range (partial range) (11, 12) and, by means of one or more second devices (21), calcium phosphate layers (CaP layers) are applied in different dose applications which create calcium phosphate layers within a range of 1-3000 nanometers, and/or
b) on implant fixtures belonging to said range with applied calcium phosphate layers, or to another range (partial range) which lacks calcium phosphate layers, one or more third devices (22) apply the bone-growth-stimulating agents or substances, for example rhEMP-2 or rhBMP-7, in different dose applications, for example in dose applications obtained by means of ca. 0.05-50 μg of bone-growth-stimulating agents or substance(s).

7. An arrangement for supplying implant fixtures made of titanium and with surfaces provided with surface structures and coated with bone-growth-stimulating and/or bone-growth-maintaining agents or substance(s), characterized by equipment for producing surface structures and a dose or doses of agents or substances on or in the respective implant/fixture, ordering equipment for transmitting orders for implant fixtures with defined surface structures and doses, equipment for receiving the ordered implant fixtures, production equipment for producing implant fixtures with defined surface structure, and application equipment for applying layers or doses of calcium phosphate and/or bone-growth-stimulating agents or substance(s), for example rhBMP-2 or rhEMP-7, on the surface structure or surface structures of the respective implant/fixture.

8. The arrangement according to patent claim 7, characterized in that the ordering equipment is intended to work with order information which includes the surface structure configuration, dose type and dose strength.

9. The arrangement as claimed in patent claim 7 or 8, characterized in that a payment function for purchasing the implant fixtures can be established via the telecommunications and/or computer network, e.g. the public telecommunications and or computer networks, which include the Internet.

10. The arrangement as claimed in patent claim 2, characterized in that the third devices are intended to coat the relevant fixture surface(s) with ca. 5 μg calcium phosphate layers rhBMP-2 or rhBMP-7.

11. The arrangement as claimed in claim 2, characterized in that it comprises surface structures charged with a dose of less than 50 μg.

12. The arrangement as claimed in claim 3, characterized in that it comprises surface structures charged with a dose of less than 50 μg.

13. The arrangement according to claim 2 characterized in that it comprises surface structures with porous oxide layers with more or less open pores charged with CaP layers within the range of 1-3000 nanometers and treated with a dose or doses of bone-growth-stimulating substance.

14. The arrangement according to claim 3, characterized in that it comprises surface structures with porous oxide layers with more or less open pores charged with CaP layers within the range of 1-3000 nanometers and treated with a dose or doses of bone-growth-stimulating substance.

15. The arrangement according to claim 4, characterized in that it comprises surface structures with porous oxide layers with more or less open pores charged with CaP layers within the range of 1-3000 nanometers and treated with a dose or doses of bone-growth-stimulating substance.

16. The arrangement as claimed in patent claim 8, characterized in that a payment function for purchasing the implant fixtures can be established via the telecommunications and/or computer network, e.g. the public telecommunications and or computer networks, which include the Internet.
